(19) United States
(12) Patent Application Publication
(10) Pub. No.: US 2025/0259701 A1
(43) Pub. Date: Aug. 14, 2025

(54) METHODS AND SYSTEMS FOR IDENTIFYING GENE VARIANTS

(71) Applicant: Illumina, Inc., San Diego, CA (US)

(72) Inventors: Vitor Ferreira Onuchic, San Diego, CA (US); Christine Amalachukwu Anyansi, San Diego, CA (US); Massimiliano Rossi, Atlanta, GA (US); Xiao Chen, Richland, VA (US); Michael Eberle, Oceanside, CA (US); Eric Edward Roller, San Diego, CA (US)

(21) Appl. No.: 18/866,956

(22) PCT Filed: Jun. 5, 2023
(86) PCT No.: PCT/US2023/024465

§ 371 Date: Nov. 18, 2024

Related U.S. Application Data
(60) Provisional application No. 63/349,993, filed on Jun. 7, 2022.

(51) Int. Cl. G16B 20/20; G16B 20/10; G16B 30/10

(52) U.S. Cl. CPC G16B 20/20; G16B 20/10; G16B 30/10

(57) ABSTRACT

Disclosed herein are systems, devices, and methods for identifying recombinant variants (such as gene conversion variants) of genes such as RHD gene and RHCE gene, the copy numbers of recombinant variants, and gene variant status (for example, heterozygous or homozygous). In some embodiments, the disclosed systems, devices, and methods include steps of receiving sequence reads which align to a RHD gene or a RHCE gene, estimating a combined copy number of a RHD gene and a RHCE gene, estimating copy numbers of a RHD-specific base and a RHCE-specific base at each of a plurality of pre-determined differentiating sites of the RHD gene and the RHCE gene, and calculating a probability of a RHCE*CE-D(2)-CE gene conversion in the nucleic acid sample.

### INCORPORATION BY REFERENCE TO ANY PRIORITY APPLICATIONS

This application claims priority to U.S. Provisional Application No. 63/349,993, filed Jun. 7, 2022, which is hereby incorporated by reference in its entirety.

### BACKGROUND

### Field

The disclosed technology relates to the field of nucleic acid sequencing. More particularly, the disclosed technology relates to detecting a RHCE*CE-D(2)-CE gene conversion event in a nucleic acid sample.

### Description of the Related Art

Rhesus (Rh) antigens play an important role in Red Blood Cells (RBC) antigens phenotype. There are over 330 RBC antigens. Variation in RBC antigens may result from variation within the RHD (Rh Blood Group D Antigen) and RHCE (Rh Blood Group CcEe Antigen) genes. Many different duplications, deletions, translocations and gene conversion events within the RHD and RHCE genes have been documented in the population, including the RHCE*CE-D(2)-CE gene conversion event.

### SUMMARY

In one aspect, disclosed herein are systems and computer-implemented methods of detecting a RHCE*CE-D(2)-CE gene conversion event in a nucleic acid sample. In some embodiments, the methods include receiving sequence reads which align to a RHD gene or a RHCE gene, estimating a combined copy number of a RHD gene and a RHCE gene in the nucleic acid sample, estimating copy numbers of a RHD-specific base and a RHCE-specific base at each of a plurality of pre-determined differentiating sites of the RHD gene and the RHCE gene, and calculating a probability of a RHCE*CE-D(2)-CE gene conversion in the nucleic acid sample based on the estimated combined copy number of the RHD gone and RHCE gene and the estimated copy numbers of the RHD-specific and RHCE-specific bases at each of the plurality of pre-determined differentiating sites.

In some embodiments, the RHCE*CE-D(2)-CE gene conversion results in a first breakpoint. In some embodiments, the plurality of pre-determined differentiating sites includes at least two pre-determined differentiating sites flanking the first breakpoint. In some embodiments, the method further includes identifying one or more sequence reads which span the first breakpoint and which include a RHD-specific base at a first pre-determined differentiating site flanking the first breakpoint and a RHCE-specific base at a second pre-determined differentiating site flanking the first breakpoint.

In some embodiments, the RHCE*CE-D(2)-CE gene conversion results in a second breakpoint. In some embodiments, the plurality of pre-determined differentiating sites includes at least two pre-determined differentiating sites flanking the second breakpoint. In some embodiments, the method includes identifying one or more sequence reads which span the second breakpoint and which include a RHD-specific base at a first pre-determined differentiating site flanking the second breakpoint and a RHCE-specific base at a second pre-determined differentiating site flanking the second breakpoint.

In some embodiments, estimating copy numbers of a RHD-specific base and a RHCE-specific base at each of a plurality of pre-determined differentiating sites of the RHD and the RHCE genes includes counting sequence reads which include a RHD-specific base at a pre-determined differentiating site among the plurality of pre-determined differentiating sites, and counting sequence reads which include a RHCE-specific base at the pre-determined differentiating site.

In some embodiments, calculating a probability of a RHCE*CE-D(2)-CE gene conversion includes estimating a gene-specific copy number at each pre-determined differentiating site of the plurality of pre-determined differentiating sites based on a proportion of sequence reads comprising a RHD-specific or RHCE-specific base at the pre-determined differentiating site multiplied by the estimated combined copy number of the RHD and RHCE genes. In some embodiments, calculating a probability of a RHCE*CE-D(2)-CE gene conversion includes detecting changes to the gene-specific copy number in consecutive pre-determined differentiating sites.

In some embodiments, estimating a combined copy number of the RHD and RHCE genes includes counting sequence reads which align to the RHD or RHCE genes. In some embodiments, estimating the combined copy number includes normalizing the count of the sequence reads which align to the RHD or RHCE genes and applying a Gaussian Mixture model. In some embodiments, the method accounts for an opposite orientation of the RHD and the RHCE genes.

In some embodiments, the plurality of pre-determined differentiating sites are identified by a method comprising identifying single-base differences between the sequence of the RHD and RHCE genes in a reference sequence, and selecting, as differentiating sites, single-base differences which are fixed across a population. In some embodiments, selecting, as differentiating sites, single-base differences which are fixed across a population comprises, for a plurality of nucleic acid samples, receiving a plurality of sequence reads which align to the RHD and RHCE genes, for each of the plurality of nucleic acid samples, estimating a gene-specific copy number for the RHD gene and a copy number for the RHCE gene, selecting a subset of nucleic acid samples of the plurality of nucleic acid samples, wherein the subset of nucleic acid samples comprises nucleic acid samples which are estimated to be diploid for the RHD gene and diploid for the RHCE gene, and selecting single-base differences which have copy numbers consistent with diploidy for the RHD gene and the RHCE gene in at least 90% of the nucleic acid samples of the subset of nucleic acid samples.

In some embodiments, the method further includes constructing one or more candidate haplotypes. In some embodiments, the one or more candidate haplotypes cover a breakpoint region of the RHCE*CE-D(2)-CE gene conversion. In some embodiments, constructing one or more candidate haplotypes includes phasing the pre-determined differentiating sites using sequence reads aligned to the RHD or RHCE gene. In some embodiments, phasing the pre-determined differentiating sites includes constructing one or more candidate haplotypes based on all sequenced bases at a first pre-determined differentiating site, and extending the one or more candidate haplotypes to a second pre-determined differentiating site by aligning sequence reads of the RHD or RHCE gene. In some embodiments, the first and second pre-determined differentiating sites flank a breakpoint of the RHCE*CE-D(2)-CE gene conversion.

In some embodiments, the methods disclosed herein further include making a variant call at a pre-determined differentiating site of the plurality of pre-determined differentiating sites. In some embodiments, the methods disclosed herein further include making a variant call for the RHCE*CE-D(2)-CE gene conversion. In some embodiments, the variant call includes a homozygous or heterozygous variant call. In some embodiments, the method further includes creating a file including a variant call.

In some embodiments, the pre-determined differentiating sites comprise a site corresponding to a position selected from chr1:25405587, chr1:25405596, chr1:25409676, or chr1:25409958 of reference genome hg38.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A schematically illustrates a RHCE*CE-D(2)-CE gene conversion event.

FIG. 1B schematically illustrates a differentiating site between RHD and RHCE genes.

FIG. 1C schematically illustrates sequence reads which align to a RHD gene or a RHCE gene and which cover a differentiating site.

FIG. 2 is a block diagram that schematically illustrates methods of detecting a RHCE*CE-D(2)-CE gene conversion event in a nucleic acid sample.

FIG. 3A is a block diagram of an exemplary sequencing system that may be used to perform the disclosed methods.

FIG. 3B is a block diagram of an exemplary computing device that may be used in connection with the exemplary sequencing system of FIG. 3A.

FIG. 4 is a graph illustrating a reduction in false negatives (FN) after implementing an embodiment of a method described herein.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### RHCE*CE-D(2)-CE

Accurate blood typing is necessary for safe blood transfusion. Basic blood typing, performed by serology, is the current standard of care (ABO/Rh+ or Rh−) and can generally be sufficient to avoid complications with most blood transfusions. However, patients requiring recurrent blood transfusions (such as patients suffering from cancer, sickle cell disease, or alpha thalassemia) can benefit from a more comprehensive assessment of their blood antigens. While serology can be used for such extended blood typing, it is dependent on the availability of antibodies specific for each blood group and can become cumbersome and expensive. Molecular blood typing, based on a patient's DNA, can be an alternative for a more complete profiling of blood antigens.

The Rhesus (Rh) factor is a widely used protein-based blood group system, second only to the ABO blood group. The antigens for the Rh blood group originate from two genes, RHD and RHCE, which are paralogous genes with around 97% identity to one another. Although most people are either Rh+ (have an active copy of RHD) or Rh− (do not have copies of RHD), a grey area exists in the form of a plethora of RHD variants: the so-called weak D, partial D, and DEL phenotypes. Aside from changes in copy number of the complete RHD or RHCE genes, two mechanisms may cause the formation of D variants: small variants leading to amino acid changes, and gene conversion, where a part of one gene is replaced by the other.

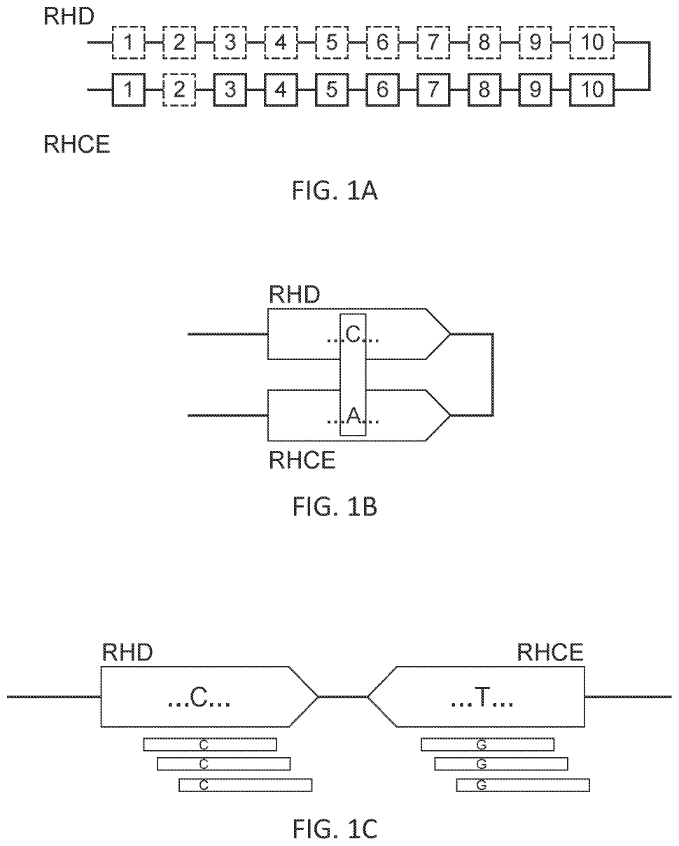

Detection of variants in RHD/RHCE can be complicated by the high sequence similarity observed between the two genes, and by the variable total copy number observed in such genes. Gene reads of the RHD/RHCE genes can, in some cases, be misaligned to the wrong gene or can be mapped with equal confidence to both genes, leading to low mapping quality. A RHCE*CE-D(2)-CE gene conversion event is a gene conversion of Exon 2 of the RHCE gene. In the RHCE*CE-D(2)-CE gene conversion event, Exon 2 of the RHCE gene is replaced with a copy of Exon 2 of the RHD gene, as illustrated in FIG. 1A.

As illustrated in FIGS. 1B and 1C, the RHD and RHCE genes are paralogs, oriented in opposite orientations in the patient's genome. Moreover, the RHCE*CE-D(2)-CE gene conversion event is not the only potential mutation in these genes. Other duplication, deletion, translocation and gene conversion events in the RHD and RHCE genes have been observed in the population. Thus, it may be difficult to detect RHCE*CE-D(2)-CE gene conversion events when sequencing the RHD and RHCE genes in a nucleic acid sample due to the high homology between the RHCE and RHD genes. For example, a RHCE*CE-D(2)-CE gene conversion may go undetected, resulting in a false negative when calling SNP variants in a nucleic sample from a patient. Embodiments of the invention overcome these challenges as described more fully below.

### Overview

Described herein are methods and systems for detecting RHCE*CE-D(2)-CE gene conversion events in a nucleic acid sample taken from a patient. The disclosed systems and methods for detecting a RHCE*CE-D(2)-CE gene conversion event in a nucleic acid sample were found to improve the specificity and sensitivity of detecting RHCE*CE-D(2)-CE gene conversions and of variant calling in the RHD and/or RHCE regions in the nucleic acid sample.

In some embodiments, the disclosed systems and methods include receiving sequence reads which align to a RHD or a RHCE gene. Once the sequence reads are received, a combined copy number of the RHD and RHCE genes in the nucleic acid sample can be estimated. Estimating the combined copy number may include counting the sequence reads that align to either RHD or RHCE regions.

The disclosed systems and methods may then estimate the copy numbers of a RHD-specific base and a RHCE-specific base at each of a plurality of pre-determined differentiating sites of the RHD and RHCE genes. These pre-determined differentiating sites may include positions in the nucleic acid sequence of the RHD or RHCE gene which include at least one base that differs between the RHD and RHCE genes, and which difference is pre-determined to be fixed in a population. Thus, these pre-determined differentiating sites may be used to determine whether a particular sequence read came from either the RHD or RHCE gene, including a RHCE*CE-D(2)-CE gene conversion event.

FIG. 1B illustrates an example of one such site which differs between the RHD and RHCE genes. In some embodiments, the differentiating sites are “pre-determined”, meaning they have been identified (such as with population studies) prior to performing the methods or implementing the systems described herein to detect the RHCE*CE-D(2)-CE gene conversion event. In some embodiments, the process for detecting the RHCE*CE-D(2)-CE gene conversion event includes counting sequence reads which include a RHD-specific base at a pre-determined differentiating site and counting sequence reads which include a RHCE-specific base at the pre-determined differentiating site. The sequence read counts may be used to estimate an RHD-specific and an RHCE-specific copy number at each of the pre-determined differentiating sites.

In some embodiments, the disclosed systems and methods include a process of calling variants related to a RHCE*CE-D(2)-CE gene conversion in the nucleic acid sample based on the copy number support for each observed base the pre-determined differentiating sites. For example, the method may include calculating a probability of a RHCE*CE-D(2)-CE gene conversion in the nucleic acid sample based on the estimated copy number supporting either the RHD-specific base or the RHCE-specific base at each of the plurality of pre-determined differentiating sites, and based on the estimated combined copy number of the RHD gene and RHCE gene. For example, a probability of a RHCE*CE-D(2)-CE gene conversion in the nucleic acid sample may be inferred by observing changes in estimated copy number of RHD-specific and RHCE-specific bases over consecutive pre-determined differentiating sites in the sequenced nucleic acids from the patient.

To further detect a RHCE*CE-D(2)-CE gene conversion event, one or more candidate haplotypes may be constructed, including candidate haplotypes which cover a breakpoint region of the RHCE*CE-D(2)-CE gene conversion. Candidate haplotypes may be constructed by, for example, phasing the pre-determined differentiating sites using sequence reads aligned to the RHD or RHCE gene.

To further detect a RHCE*CE-D(2)-CE gene conversion event, the methods and systems disclosed herein may include identifying one or more sequence reads which span a breakpoint of a RHCE*CE-D(2)-CE gene conversion event and which include a RHD-specific base at a first pre-determined differentiating site flanking the breakpoint and a RHCE-specific base at a second pre-determined differentiating site flanking the breakpoint.

The disclosed systems and methods can improve the recall (also known as sensitivity, the percentage of true variants that are correctly detected) of single nucleotide polymorphisms (SNPs) generated by a RHCE*CE-D(2)-CE gene conversion event by 20%, 50%, 80%, 100% or more, for example by reducing false negatives.

### Definitions

Unless defined otherwise, technical and scientific terms used herein have the same meaning as commonly understood by one of ordinary skill in the art to which the present disclosure belongs. See, for example, Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “base” or “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties,

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

A “genome” refers to the complete genetic information of an organism or virus, expressed in nucleic acid sequences.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. In various embodiments, the reference sequence is significantly larger than the reads that are aligned to it. For example, it may be at least about 100 times larger, or at least about 1000 times larger, or at least about 10,000 times larger, or at least about 10⁵ times larger, or at least about 10⁶ times larger, or at least about 10⁷ times larger. In one example, the reference sequence is that of a full-length genome. Such sequences may be referred to as genomic reference sequences. For example, the reference sequence can be a reference human genome sequence, such as hg19 or hg38. In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), etc., of any species. In various embodiments, the reference sequence is a consensus sequence or other combination derived from multiple individuals. However, in certain applications, the reference sequence may be taken from a particular individual.

The term “nucleic acid sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments the nucleic acid sample comprises at least one nucleic acid sequence whose copy number is suspected of having undergone variation. Such samples may include, but are not limited to sputum/oral fluid, amniotic fluid, blood, a blood fraction, or fine needle biopsy samples (such as surgical biopsy, fine needle biopsy, etc.), urine, peritoneal fluid, pleural fluid, and the like. Although the sample is often taken from a human subject (such as a patient), the sample may be from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (such as namely, a sample that is not subjected to any such pretreatment method(s)). Such “treated” or “processed” samples are still considered to be biological “test” samples with respect to the methods described herein.

The term “read” or “sequence read” (or sequencing reads) refer to a sequence obtained from a portion of a nucleic acid sample. A read may be represented by a string of nucleotides sequenced from any part or all of a nucleic acid molecule. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (such as at least about 25 bp) that can be used to identify a larger sequence or region, for example, that can be aligned and specifically assigned to a chromosome or genomic region or gene. For example, a sequence read may be a short string of nucleotides (such as 20-150 bases) sequenced from a nucleic acid fragment, a short string of nucleotides at one or both ends of a nucleic acid fragment, or the sequencing of the entire nucleic acid fragment that exists in the biological sample. Sequence reads may be obtained by any method known in the art. For example, a sequence read may be obtained in a variety of ways, such as using sequencing techniques or using probes, such as in hybridization arrays or capture probes, or amplification techniques, such as the polymerase chain reaction (PCR) or linear amplification using a single primer or isothermal amplification. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The term “sequencing depth,” as used herein, generally refers to the number of times a locus is covered by a sequence read aligned to the locus. The locus may be as small as a nucleotide, or as large as a chromosome arm, or as large as the entire genome. Sequencing depth can be expressed as 50×, 100×, etc., where “×” refers to the number of times a locus is covered with a sequence read. Sequencing depth can also be applied to multiple loci, or the whole genome, in which case x can refer to the mean number of times the loci or the haploid genome, or the whole genome, respectively, is sequenced. When a mean depth is quoted, the actual depth for different loci included in the dataset spans over a range of values. Ultra-deep sequencing can refer to at least 100× in sequencing depth.

As used herein, the terms “aligned,” “alignment,” or “aligning” refer to the process of comparing a read or tag to a reference sequence and thereby determining the likelihood of the reference sequence contains the read sequence. If the reference sequence contains the read, the read may be mapped to the reference sequence or, in certain embodiments, to a particular location in the reference sequence. For example, the alignment of a read to the reference sequence for human chromosome 13 will tell the likelihood of the read is present in the reference sequence for chromosome 13. In some cases, an alignment additionally indicates a location where the read or tag maps to in the reference sequence. For example, if the reference sequence is the whole human genome sequence, an alignment may indicate that a read is present on chromosome 13, and may further indicate that the read is on a particular strand and/or site of chromosome 13. A “site” may be a unique position on a polynucleotide sequence or a reference genome (i.e. chromosome ID, chromosome position and orientation). In some embodiments, a site may provide a position for a residue, a sequence tag, or a segment on a sequence.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. The matching of a sequence read in aligning can be a 100% sequence match or less than 100% (non-perfect match).

Alignment may be performed by modifications and/or combinations of methods such as Burrows-Wheeler Aligner (BWA), iSAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The term “mapping” used herein refers to specifically assigning a sequence read to a larger sequence, such as a reference genome, by alignment.

A “genetic variation” or “genetic alteration” refers to a particular genotype present in certain individuals, and often a genetic variation is present in a statistically significant sub-population of individuals. The presence or absence of a genetic variance can be determined using a method or apparatus described herein. In certain embodiments, the presence or absence of one or more genetic variations is determined according to an outcome provided by methods and apparatuses described herein. In some embodiments, a genetic variation is a chromosome abnormality (such as aneuploidy), partial chromosome abnormality or mosaicism, each of which is described in greater detail herein. Non-limiting examples of genetic variations include one or more deletions (such as micro-deletions), duplications (such as micro-duplications), insertions, mutations, polymorphisms (such as single-nucleotide polymorphisms), fusions, repeats (such as short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 1,000 kilobases (kb) in length (for example about 10 bp, 50 bp, 100 bp, 500 bp, 1 kb, 5 kb, 10 kb, 50 kb, 100 kb, 500 kb, or 1000 kb in length).

A genetic variation is sometimes a deletion. In certain embodiments a deletion is a mutation (such as a genetic aberration) in which a part of a chromosome or a sequence of DNA is missing. A deletion is often the loss of genetic material. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion. A deletion can comprise the deletion of a single base.

A genetic variation is sometimes a genetic duplication. In certain embodiments a duplication is a mutation (such as a genetic aberration) in which a part of a chromosome or a sequence of DNA is copied and inserted back into the genome. In certain embodiments a genetic duplication (i.e. duplication) is any duplication of a region of DNA. In some embodiments a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication. A duplication sometimes comprises one or more copies of a duplicated nucleic acid. A duplication sometimes is characterized as a genetic region repeated one or more times (such as repeated 1, 2, 3, 4, 5, 6, 7, 8, 9 or 10 times). Duplications can range from small regions (thousands of base pairs) to whole chromosomes in some instances, Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation is sometimes an insertion. An insertion is sometimes the addition of one or more nucleotide base pairs into a nucleic acid sequence. An insertion is sometimes a microinsertion. In certain embodiments an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (i.e., insertion) of nucleic acid of unknown origin into a genome, chromosome, or segment thereof. In certain embodiments an insertion comprises the addition (i.e. insertion) of a single base.

A genetic variation sometimes includes copy number variations, i.e., variations in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A copy number variant may refer to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations may include deletions, including microdeletions, insertions, including microinsertions, duplications, multiplications, and translocations. CNVs encompass chromosomal aneuploidies and partial aneuploidies.

### Embodiments of Methods and Systems of Detecting the RHCE*CE-D(2)-CE Gene Conversion Event

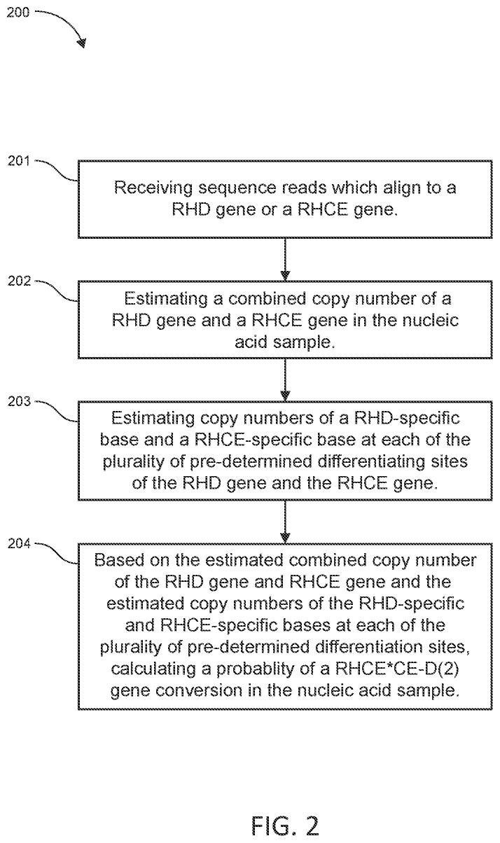

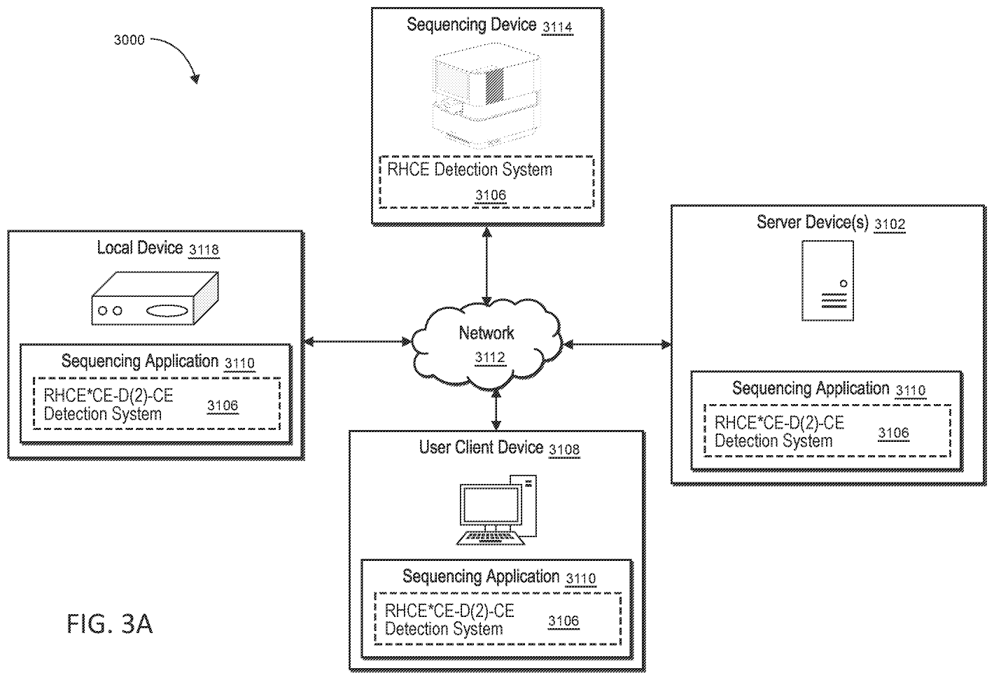

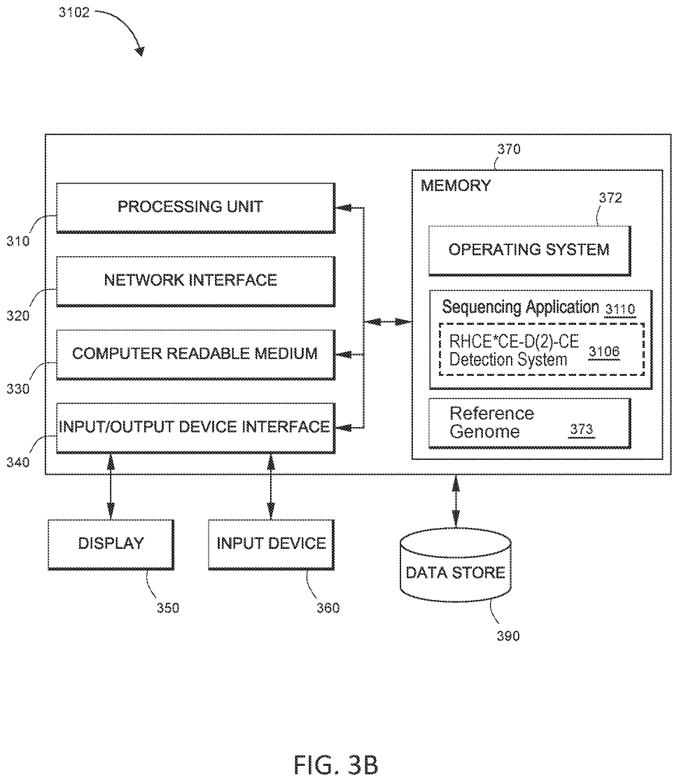

FIG. 2 is a block diagram that schematically illustrates an exemplary method 200 of detecting a RHCE*CE-D(2)-CE gene conversion event in a nucleic acid sample. In some embodiments, the method 200 is implemented on a computer. The method 200 may be embodied in a set of executable program instructions stored on a computer-readable medium, such as one or more disk drives, of a computing system. For example, the server device 3102 shown in FIGS. 3A and 3B and described in greater detail below can execute a set of executable program instructions to implement the method 200. When the method 200 is initiated, the executable program instructions can be loaded into a memory, such as RAM, and executed by one or more processors of a server device 3102. Although the method 200 is described with respect to the server device 3102 shown in FIG. 3B, the description is illustrative only and is not intended to be limiting. In some embodiments, the method 200 or portions thereof may be performed serially or in parallel by multiple computing systems.

As shown in FIG. 2, the method 200 for detecting a RHCE*CE-D(2)-CE gene conversion event in a nucleic acid sample may start from block 201, wherein sequence reads which align to the RHD or RHCE gene are received. For example, sequence reads which align to the RHD or RHCE gene may be mapped to a reference sequence to determine an alignment to a RHD or RHCE gene. Next, the method 200 may proceed to block 202, wherein a combined copy number of a RHD gene and a RHCE gene in the nucleic acid sample is estimated. The method 200 may then proceed to block 203, wherein copy numbers of a RHD-specific base and a RHCE-specific base at each of a plurality of pre-determined differentiating sites of the RHD and the RHCE genes are estimated. Next, the method 200 may proceed to block 204, wherein based on the estimated copy numbers of the RHD and RHCE genes and the estimated copy numbers of each the RHD-specific and RHCE-specific bases at each of the plurality of pre-determined differentiating sites, a probability of a RHCE*CE-D(2)-CE gene conversion in the nucleic acid sample is calculated.

### Receiving Sequence Reads Which Align to the RHD Gene or the RHCE Gene

In some embodiments, the methods and systems disclosed herein include a step of receiving a plurality of sequence reads which align to the RHD gene or to the RHCE gene. In some embodiments, the sequence reads are generated from a sample obtained from a subject.

Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA). Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by whole genome sequencing (WGS). The WGS can be clinical WGS (cWGS). The sample can comprise cells, cell-free DNA, cell-free fetal DNA, amniotic fluid, a blood sample, a biopsy sample, or a combination thereof.

In some embodiments, the sequence reads are obtained by aligning the reads to the RHD or RHCE regions of a reference sequence. In some embodiments, the sequence reads are obtained by aligning a first plurality of sequence reads generated from a sample to a reference genome sequence to obtain a second plurality of sequence reads which align to the RHD gene or to the RHCE gene in the reference genome sequence. In some embodiments, a computing system stores the first plurality of sequence reads in memory. The computing system may load the first plurality of sequence reads into memory. A sequence read can be aligned to RHD gene or RHCE gene in the reference sequence with an alignment quality score of zero or more. A sequence read can be aligned to RHD gene or RHCE gene in the reference sequence with an alignment quality score of about zero (for example, when a sequence is aligned to a region where the gene and the gene paralog are highly homologous).

In some embodiments, the sequence reads are obtained from a file containing sequencing information. In some embodiments, the file is on a computer storage medium (such as a computer hard drive, for example a spinning magnetic disk drive or a solid state drive). In some embodiments, the file is a stored in the format of a BAM, SAM, CRAM, or VCF file. In some embodiments, the sequence reads cover a breakpoint region of the RHCE*CE-D(2)-CE gene conversion event.

### Estimating a Combined Copy Number

In some embodiments, estimating a combined copy number of the RHD and RHCE genes comprises counting sequence reads which align to the RHD or RHCE genes. In some embodiments, the combined copy number between RHD and RHCE genes is estimated by counting the total number of reads aligning to either RHD or RHCE in a reference genome sequence. In some embodiments, counting the total number of reads aligning to either RHD or RHCE in a reference genome sequence includes counting sequence reads which can be mapped with equal confidence to either the RHD or RHCE genes (leading to zero mapping quality). In some embodiments, sequence reads align to regions in both the RHD gene and the RHCE gene with a mapping quality of zero because the sequence is identical between the two regions, due to the high homology between regions of the RHD gene and the RHCE gene. In some embodiments, by counting sequence reads with a low mapping quality (including a mapping quality of zero), a combined copy number of the RHD and RHCE genes may be estimated despite the high sequence homology.

In some embodiments, estimating the combined copy number comprises normalizing the count of the sequence reads which align to the RHD or RHCE genes and applying a Gaussian mixture model. In some embodiments, the Gaussian mixture model includes a plurality of Gaussians, each representing a different integer copy number, given the normalized number of the sequence reads (for example, normalized and/or corrected sequence reads) aligned to the RHD gene or the RHCE gene. For example, the read count may be normalized by the length of the region and against a set of 3000 genomic regions of 2000 bp expected to be consistently diploid across populations. In some embodiments, a Gaussian mixture model is then used to infer the most likely copy number of RHD+RHCE genes based on the observed normalized depth signal.

The total copy number can be, for example, 2, 3, 4, 5, 6, 7, 8, 9, 10, or more copies. The Gaussian mixture model can comprise a one-dimensional Gaussian mixture model. The plurality of Gaussians of the Gaussian mixture model can represent integer copy numbers, for example, 0 to 5, 0 to 6, 0 to 7, 0 to 8, 0 to 9, 0 to 10, 0 to 11, 0 to 12, 0 to 13, 0 to 14, or 0 to 15. For example, the plurality of Gaussians of the Gaussian mixture model can represent integer copy numbers from 0 to 10. A mean of each of the plurality of Gaussians can be the integer copy number represented by the Gaussian. A mean of each of the plurality of Gaussians can be the integer copy number represented by the Gaussian (such as copy numbers of 1, 2, 3, 4, 5, 6, 7, 8, 9, 10, 11, 12, 13, 14, 15, or more). The standard deviation of a Gaussian can be or be about, for example, 0.1, 0.2, 0.3, 0.4, 0.5, 0.6, 0.7, 0.8, 0.9, 1, or more. The plurality of Gaussians of the Gaussian mixture model can comprise, for example, 5, 6, 7, 8, 9, 10, 11, 12, 13, 14, 15, 16, or more, Gaussians. For example, the plurality of Gaussians of the Gaussian mixture model can comprise 5 Gaussians.

To estimate the combined copy number of RHD gene and RHCE gene, the computing system can determine the total copy number of RHD gene and RHCE gene using a Gaussian mixture model and a predetermined posterior probability threshold, given the normalized number of the sequence reads aligned to the RHD gene or RHCE gene. The predetermined posterior probability threshold can be, for example, 0.7, 0.75, 0.8, 0.85, 0.95, or more.

### Estimating Gene-Specific Base Copy Numbers at Pre-Determined Differentiating Sites

In some embodiments, the methods and systems disclosed herein include a step of estimating copy numbers of a RHD-specific base and a RHCE-specific base at each of a plurality of pre-determined differentiating sites of the RHD gene and the RHCE gene.

In some embodiments, sequence information (such as basecalls) are assessed at one or more pre-determined differentiating sites. As used herein, “pre-determined differentiating sites” refers to sites in a nucleic acid sequence which are different between the sequence of the RHD and RHCE genes. A pre-determined differentiating site may be fixed in the population, for example, and observed as a base difference between the RHD and RHCE genes in at least 90%, at least 95%, at least 98%, or at least 99% of the population. In some embodiments, the RHCE*CE-D(2)-CE gene conversion results in a first breakpoint, and the plurality of pre-determined differentiating sites comprises at least two pre-determined differentiating sites flanking the first breakpoint. In some embodiments, the plurality of pre-determined differentiating sites may include a site corresponding to a position selected from chr1:25405587, chr1:25405596, chr1:25409676, or chr1:25409958 of reference genome hg38, (for example, available at GenBank assembly accession GCA_000001405.15).

In some embodiments, the proportion of reads supporting an expected RHD-specific base and the RHCE-specific base is assessed at each of the pre-determined differentiating sites of the plurality of pre-determined sites. For example, sequence reads which include a RHD-specific base at a pre-determined differentiating site among the plurality of pre-determined differentiating sites may be counted, and sequence reads which include a RHCE-specific base at the pre-determined differentiating site may be counted. The count may be normalized using methods described with reference to estimating a combined copy number of RHD and RHCE genes.

A computing system (such as server device 3102) can determine a normalized number of the sequence reads which contain a RHD-specific or RHCE-specific base at a given pre-determined differentiating site. To determine the normalized number of the sequence reads which contain a RHD-specific or RHCE-specific base, the computing system can determine the normalized number of the sequence reads including a RHD-specific or a RHCE-specific base at a pre-determined differentiating site using (1a) a depth of the sequence reads aligned to the pre-determined differentiating site and containing a RHD-specific or a RHCE-specific base, (1b) a length of the pre-determined differentiating site, (2a) a depth of sequence reads which align to regions in the RHD or RHCE gene not including the pre-determined differentiating site, and/or (2b) a length of each of the regions in the RHD or RHCE gene not including the pre-determined differentiating site.

### Estimating a Probability of a RHCE*CE-D(2)-CE Gene Conversion Event

In some embodiments, the methods and systems disclosed herein include a step of, based on the estimated combined copy number of the RHD gene and RHCE gene and the estimated copy numbers of the RHD-specific and RHCE-specific bases at each of the plurality of pre-determined differentiating sites, calculating a probability of a RHCE*CE-D(2)-CE gene conversion in the nucleic acid sample.

In some embodiments, a gene-specific copy number (such as a copy number for each of the RHD and RHCE genes) is estimated for each pre-determined differentiating site of the plurality of pre-determined differentiating sites. The gene-specific copy number may be based on a proportion of sequence reads comprising a RHD-specific or RHCE-specific base at the pre-determined differentiating site. In some embodiments, the method includes multiplying the proportion of sequence reads supporting the RHD-specific base or RHCE-specific base at each pre-determined differentiating site by the estimated combined copy number, thereby estimating a gene-specific copy number at each pre-determined differentiating site. The gene-specific copy number can be for example, 0, 1, 2, 3, 4 or more. The gene-specific copy number may be an integer.

In some embodiments, the method includes detecting changes to a gene-specific copy number (such as changes in the proportion of reads supporting a RHD-specific or a RHCE-specific base) in consecutive pre-determined differentiating sites. and estimating the probability of a RHCE*CE-D(2)-CE gene conversion event. For example, if a portion of either RHD or RHCE genes has been replaced with the corresponding region from the other gene, this would lead to an increase or decrease in the proportion of reads supporting the RHD-specific and RHCE-specific bases at the pre-determined differentiating sites.

In some embodiments, to determine the likelihood of a RHCE*CE-D(2)-CE gene conversion, a computing system can, for one or more pairs of consecutive pre-determined differentiating sites of the plurality of pre-determined differentiating sites, determine a copy number of the RHCE-specific bases at the consecutive pre-determined differentiating sites given (1) a number of sequence reads aligned to the RHD gene or RHCE gene each comprising two or more RHCE-specific bases at the consecutive pre-determined differentiating sites, (2) a number of sequence reads aligned to the RHD gene or RHCE gene each comprising a RHCE-specific base and a RHD-specific base, or the RHD-specific base and the RHCE-specific base at the consecutive pre-determined differentiating sites, and/or (3) a number of sequence reads aligned to the RHD gene or RHCE gene each comprising the RHCE bases at the consecutive pre-determined differentiating sites.

### Identifying Sequence Reads Spanning a Breakpoint

In some embodiments, the methods and systems disclosed herein include a step of identifying one or more sequence reads that span the first breakpoint and which include a RHD-specific base at a first pre-determined differentiating site flanking the first breakpoint and a RHCE-specific base at a second pre-determined differentiating site flanking the first breakpoint. Thus, the method may include identifying, among a plurality of sequence reads which align to the RHD gene or the RHCE gene, one or more sequence reads that cover one of two breakpoints of a RHCE*CE-D(2)-CE gene conversion, and which include at least two pre-determined differentiating sites, one on either side of the breakpoint, with a RHD-specific base at the first pre-determined differentiating site flanking the breakpoint, and a RHCE-specific base at the second pre-determined site flanking the breakpoint.

In some embodiments, the RHCE*CE-D(2)-CE gene conversion results in a second breakpoint, and the plurality of pre-determined differentiating sites comprises at least two pre-determined differentiating sites flanking the second breakpoint. In some embodiments, the method further includes identifying one or more sequence reads which span the second breakpoint and which include a RHD-specific base at a first pre-determined differentiating site flanking the second breakpoint and a RHCE-specific base at a second pre-determined differentiating site flanking the second breakpoint. Thus, in some embodiments, the method includes identifying, for each of two breakpoints of a RHCE*CE-D(2)-CE gene conversion, one or more sequence reads which span each breakpoint and which include a RHD-specific base at a first pre-determined differentiating site flanking the breakpoint and a RHCE-specific base at a second pre-determined differentiating site flanking the breakpoint.

In some embodiments, a pre-determined differentiating site flanking a breakpoint is selected from a site corresponding to a position selected from chr1:25405587, chr1:25405596, chr1:25409676, or chr1:25409958 of reference genome hg38.

### Constructing Candidate Haplotypes

In some embodiments, methods and systems disclosed herein further include a step of constructing one or more candidate haplotypes. In some embodiments, the one or more candidate haplotypes cover a breakpoint region of the RHCE*CE-D(2)-CE gene conversion.

In some embodiments, constructing one or more candidate haplotypes comprises phasing the pre-determined differentiating sites using sequence reads aligned to the RHD or RHCE gene. In some embodiments, phasing the pre-determined differentiating sites includes constructing one or more candidate haplotypes based on all sequenced bases at a first pre-determined differentiating site, and extending the one or more candidate haplotypes to a second pre-determined differentiating site by aligning sequence reads of the RHD or RHCE gene.

For example, candidate haplotypes may be formed from all sequenced bases at the first pre-determined differentiating site. For example, two candidate haplotypes may be formed if two bases are possible at a first pre-determined differentiating site based on basecalls from sequencing reads covering the first pre-determined differentiating site. In some embodiments, the haplotypes are then extended to the next pre-determined differentiating site by considering all sequencing reads that can be uniquely assigned to a single candidate haplotype. In some embodiments, if these sequencing reads support only a single base at the next differentiating site for a given candidate haplotype, then the haplotype is extended with that base. In some embodiments, when a candidate haplotype can be extended by two possible bases at the second pre-determined differentiating site, then both possible extended haplotypes are included in the set of candidate haplotypes, growing the set by 1. In some embodiments, subsequent extension steps are performed at a third pre-determined differentiating site, and the steps may be repeated until all sites have been processed. In some embodiments, this process yields a set of candidate haplotypes based on the bases observed at the plurality of pre-determined differentiating sites.

In some embodiments, a computing system constructs one or more candidate haplotypes originating from RHD gene or RHCE gene in a region of the RHCE gene, comprising a plurality of pre-determined differentiating sites using sequence reads aligned to the RHD gene or RHCE gene, comprising the plurality of pre-determined differentiating sites. For example, a sequence read can be aligned to the reference sequence such that the sequence read overlaps a pre-determined differentiating site. A sequence read can be aligned to the region of RHD gene, or the corresponding region of the RHCE gene, comprising the plurality of pre-determined differentiating sites with an alignment quality score of zero or more.

In some embodiments, the one or more candidate haplotypes comprises a wildtype RHD haplotype, a wildtype RHCE haplotype, and/or a RHCE*CE-D(2)-CE haplotype. A RHCE*CE-D(2)-CE haplotype can include both RHD bases and RHCE bases. A RHCE*CE-D(2)-CE haplotype can be a recombinant variant. The RHCE*CE-D(2)-CE haplotype can comprise a RHCE variant haplotype. A haplotype can comprise a reciprocal recombinant variant. A haplotype can comprise a non-reciprocal recombinant variant or a gene conversion variant. The reference sequence can comprise a reference genome sequence.

To phase the one or more haplotypes originating from RHD gene or RHCE gene, the computing system can analyze linkage information between the pre-determined differentiating sites of the plurality of pre-determined differentiating sites using sequence reads aligned to the RHD or RHCE region, comprising the plurality of pre-determined differentiating sites. To phase the one or more haplotypes originating from RHD gene or RHCE gene, the computing system can phase the one or more haplotypes originating from RHD gene or RHCE gene using sequence reads aligned to two or more of the plurality of pre-determined differentiating sites.

In some embodiments, the first and second pre-determined differentiating sites may flank a breakpoint of the RHCE*CE-D(2)-CE gene conversion. In some embodiments, a pre-determined differentiating site flanking a breakpoint is selected from a site corresponding to a position selected from chr1:25405587, chr1:25405596, chr1:25409676, or chr1:25409958 of reference genome hg38.

For example, the boundaries of the RHCE*CE-D(2)-CE gene conversion event may be confirmed by phasing of pre-determined differentiating sites using sequencing reads mapped to either the RHD or RHCE genes over each breakpoint region. In some embodiments, the method further includes confirming a RHCE*CE-D(2)-CE gene conversion by identifying sequencing reads or sequencing read pairs which span a RHCE*CE-D(2)-CE breakpoint and which contain a RHD-specific base and a RHCE-specific base at consecutive pre-determined differentiating sites.

### Identifying Pre-Determined Differentiating Sites

Disclosed herein are methods and systems for identifying a plurality of pre-determined differentiating sites. In some embodiments, the method comprises identifying single-base differences between the sequence of the RHD and RHCE genes in a reference sequence. For example, a reference sequence of the RHD gene may be compared with a reference sequence of a RHCE gene by aligning the sequences to each other and noting all sites with single base differences between the two gene sequences. The positions of those differentiating sites in both RHD and RHCE genes may then be stored to an electronic storage. For example, a file may be created including a list of the single base differences.

In some embodiments, the method includes selecting, as differentiating sites, single-base differences which are fixed across a population. For example, the method may include, for a plurality of nucleic acid samples (such as a plurality of nucleic acid samples from a population of individuals), receiving a plurality of sequence reads which align to the RHD and RHCE genes. In some embodiments, the plurality of nucleic acid samples are derived from individuals of a population, such as more than 100, more than 500, more than 1,000, more than 5,000, or more than 10,000 individuals. In some embodiments, the population is a diverse population, such as a genetically diverse population including individuals from a plurality of ethnic groups, such as to account for differences in population types and increase the likelihood that single-base differences do not comprise differences due to population type. The method may further include, for each of the plurality of nucleic acid samples, estimating a gene-specific copy number for the RHD gene and a copy number for the RHCE gene. The method may further include selecting a subset of nucleic acid samples of the plurality of nucleic acid samples, wherein the subset of nucleic acid samples comprises nucleic acid samples which are estimated to be diploid for the RHD gene and diploid for the RHCE gene (such as using only the data from samples which are estimated to not contain the RHCE*CE-D(2)-CE gene conversion). The method may further include selecting single-base differences which have copy numbers consistent with diploidy for the RHD gene and the RHCE gene in at least 90%, at least 95%, at least 97%, at least 98%, or at least 99% of the nucleic acid samples of the subset of nucleic acid samples.

The method may further include creating a file which lists the positions of the selected single base differences, thereby generating a file including a plurality of pre-determined differentiating sites. In some embodiments, the file is on a computer storage medium (such as a computer hard drive, for example a spinning magnetic disk drive or a solid state drive). In some embodiments, the file is a stored in the format of a BAM, SAM, CRAM, or VCF file. The file may include information for the pre-determined differentiating sites such as the chromosome name where the pre-determined differentiating site is located, a 1-based inclusive start position in RHCE, the expected base sequences for a RHCE read mapped to the start position in RHCE, a 1-based inclusive start position in RHD, the expected base sequences for a RHD read mapped to the start position in RHD, the region of RHCE corresponding to the RHD start position, a unique name for the pre-determined differentiating site, and/or the orientation of the pre-determined differentiating site given by the orientation of the gene.

### Variant Calling

In some embodiments, the methods and systems disclosed herein further includes a step of making a variant call at a pre-determined differentiating site of the plurality of pre-determined differentiating sites. In some embodiments, variant calls are made at each pre-determined differentiating site in the gene receiving the gene conversion (i.e., the RHCE gene), with the alternative allele being the base observed in the source of the gene conversion event (i.e., the RHD gene). In some embodiments, a heterozygous or homozygous variant call is made based on the gene-specific copy number observed over each pre-determined differentiating site within the gene conversion event region.

In some embodiments, a variant call is made for the RHCE*CE-D(2)-CE gene conversion. In some embodiments, the variant call comprises a homozygous or heterozygous variant call, including at an individual pre-determined differentiating site and/or for the RHCE*CE-D(2)-CE gene conversion.

In some embodiments, the methods and systems disclosed herein further include a step of creating a file including a variant call. In some embodiments, the file is on a computer storage medium (such as a computer hard drive, for example a spinning magnetic disk drive or a solid state drive). In some embodiments, the file is a stored in the format of a BAM, SAM, CRAM, or VCF file. In some embodiments, the file is a VCF file.

### Accounting for Opposite Orientation of the RHD and RHCE Genes

In some embodiments, the RHD and RHCE genes are paralogs on opposite orientation within a genome, as depicted in the illustrations of FIG. 1B and FIG. 1C. Accordingly, in some embodiments, the methods and systems account for an opposite orientation of the RHD and the RHCE genes. In some embodiments, the opposite orientation of the RHD and RHCE genes is accounted for when counting or identifying sequence reads which include a RHD-specific base or a RHCE-specific base at a pre-determined differentiating site.

For example, in the embodiment of FIG. 1B, a pre-determined differentiating site is shown, which has a RHD-specific base “C” (cytosine) and a RHCE-specific base “A” (adenine). As shown in the embodiment of FIG. 1C, sequence reads which align to the RHD gene include a C at the pre-determined differentiating site. If a gene conversion from RHD to RHCE has occurred at the pre-determined differentiating site, sequence reads which align to the RHCE gene would be expected to include a “G” (guanine, the base-pair complement of cytosine) at the pre-determined site, as shown in FIG. 1C, due to the opposite orientation of the RHD and RHCE genes.

Accordingly, in some embodiments, estimating copy numbers of a RHD-specific base and a RHCE-specific base at each of a plurality of pre-determined differentiating sites of the RHD and the RHCE genes includes counting sequence reads which include a RHD-specific base or its complement at a pre-determined differentiating site among the plurality of pre-determined differentiating sites, and counting sequence reads which include a RHCE-specific base or its complement at the pre-determined differentiating site.

### Embodiments of Sequencing Systems

FIG. 3A illustrates a diagram of an environment in which a RHCE*CE-D(2)-CE detection system can operate in accordance with one or more implementations. The following paragraphs describe the RHCE*CE-D(2)-CE detection system with respect to illustrative figures that portray example implementations and embodiments. For example, FIG. 3A illustrates a schematic diagram of a computing system 3000 in which a RHCE*CE-D(2)-CE detection system 3106 operates in accordance with one or more implementations. As illustrated, the computing system 3000 includes one or more server device(s) 3102 connected to a user client device 3108, a local device 3118, and a sequencing device 3114 via a network 3112. The network 3112 can comprise any suitable network over which computing devices can communicate.

As shown in FIG. 3A, the computing system 3000 includes the server device(s) 3102. In various implementations, the server device(s) 3102 may generate, receive, analyze, store, and transmit digital data, such as data for nucleobase calls or sequenced nucleic-acid polymers. In some implementations, the server device(s) 3102 receive various data from the sequencing device 3114, such as data from a sample genome and/or sequence reads. The server device(s) 3102 may also communicate with the user client device 3108. In particular, the server device(s) 3102 can send data for sequence reads, direct nucleobase calls, nucleobase calls, and/or sequencing metrics to the user client device 3108.

As shown, the server device(s) 3102 includes a sequencing application 3110. In general, the sequencing application 3110 analyzes the data (such as call data) received from the sequencing device 3114 or elsewhere to determine nucleobase sequences for nucleic-acid polymers. For example, the sequencing application 3110 can receive raw data from the sequencing device 3114 and determine a nucleobase sequence for a sample genome or a nucleic-acid segment. In some implementations, the sequencing application 3110 determines the sequences of nucleobases in DNA and/or RNA segments or oligonucleotides.

As also shown, the sequencing application 3110 includes the RHCE*CE-D(2)-CE detection system 3106. As described below, the RHCE*CE-D(2)-CE detection system 3106 can detect a RHCE*CE-D(2)-CE gene conversion event in a nucleic acid sample. For example, in some embodiments, the RHCE*CE-D(2)-CE detection system 3106 receives sequence reads obtained from a nucleic acid sample. The RHCE*CE-D(2)-CE detection system 3106 further estimates a combined copy number of a RHD gene and a RHCE gene in the nucleic acid sample The RHCE*CE-D(2)-CE detection system 3106 further estimates copy numbers of a RHD-specific base and a RHCE-specific base at each of a plurality of pre-determined differentiating sites of the RHD gene and the RHCE gene. Based on the estimated combined copy number of the RHD gene and RHCE gene and the estimated copy numbers of the RHD-specific and RHCE-specific bases at each of the plurality of pre-determined differentiating sites, The RHCE*CE-D(2)-CE detection system 3106 can calculate a probability of a RHCE*CE-D(2)-CE gene conversion in the nucleic acid sample.

Moreover, while the RHCE*CE-D(2)-CE detection system 3106 is described being implemented on the server device(s) 3102, as part of the sequencing application 3110, in some implementations, the RHCE*CE-D(2)-CE detection system 3106 is implemented by (such as located entirely or in part) on the user client device 3108, the sequencing device 3114, and/or the local device 3118. As mentioned, in some implementations, the RHCE*CE-D(2)-CE detection system 3106 is implemented by one or more other components of the computing system 3000, such as the sequencing device 3114. In particular, the RHCE*CE-D(2)-CE detection system 3106 can be implemented in a variety of different ways across the server device(s) 3102, the network 3112, the user client device 3108, the local device 3118, and the sequencing device 3114.

As further shown in FIG. 3A, the computing system 3000 includes the user client device 3108. In various implementations, the user client device 3108 can generate, store, receive, and send digital data. In particular, the user client device 3108 can receive the data from the sequencing device 3114. As further illustrated, the user client device 3108 includes a sequencing application 3110. The sequencing application 3110 may be a web application or a native application stored and executed on the user client device 3108 (e.g., a mobile application, desktop application, or web application). The sequencing application 3110 can receive data from the sequencing application 3110 and/or RHCE*CE-D(2)-CE detection system 3106. For example, the user client device 3108 can receive variant call files and/or alignment files from the sequencing application 3110.

The sequencing application 3110 can also include instructions that (when executed) cause the user client device 3108 to receive data from the RHCE*CE-D(2)-CE detection system 3106 and present data from the sequencing device 3114 and/or the server device(s) 3102. Furthermore, the sequencing application 3110 can instruct the user client device 3108 to display data for variant calls, such as nucleobase calls or an indication of a calculated probability of a RHCE*CE-D(2)-CE gene conversion event. Indeed, the user client device 3108 can display nucleobase call results for a genome sample and/or an indication of a predicted RHCE*CE-D(2)-CE gene conversion.

As further shown in FIG. 3A, the computing system 3000 includes the sequencing device 3114. In various implementations, the sequencing device 3114 can sequence a genomic sample or other nucleic-acid polymer. For example, the sequencing device 3114 analyzes nucleic-acid segments or oligonucleotides extracted from genomic samples to generate data either directly or indirectly on the sequencing device 3114. More particularly, the sequencing device 3114 receives and analyzes, within nucleotide-sample slides (such as flow cells), nucleic-acid sequences extracted from genomic samples. In one or more implementations, the sequencing device 3114 utilizes SBS to sequence a genomic sample or other nucleic-acid polymers. In addition to, or in the alternative to communicating across the network 3112, in some implementations, the sequencing device 3114 bypasses the network 3112 and communicates directly with the user client device 3108.

As further depicted in FIG. 3A, in some implementations, the server device(s) 3102 includes a distributed collection of servers, where the server device(s) 3102 include several server devices distributed across the network 3112 and located in the same or different physical locations. For instance, the server device(s) 3102 can be implemented, in whole or in part, on the local device 3118. To illustrate, the local device 3118 may implement the sequencing application 3110 and/or the RHCE*CE-D(2)-CE detection system 3106. Further, the server device(s) 3102 and/or the local device 3118 can include a content server, an application server, a communication server, a web-hosting server, or another type of server.

The user client device 3108 illustrated in FIG. 3A can include various types of client devices. For example, in some implementations, the user client device 3108 includes non-mobile devices, such as desktop computers or servers, or other types of client devices. In various implementations, the user client device 3108 includes mobile devices, such as laptops, tablets, mobile telephones, or smartphones.

Though FIG. 3A illustrates the components of the computing system 3000 communicating via the network 3112, in certain implementations, the components of computing system 3000 can also communicate directly with each other, bypassing the network 3112. For instance, in some implementations, the user client device 3108 communicates directly with the sequencing device 3114. Additionally, in some implementations, the user client device 3108 communicates directly with the RHCE*CE-D(2)-CE detection system 3106 and/or the server device(s) 3102. In some implementations, the user client device 3108 communicates directly with the local device 3118. Moreover, the RHCE*CE-D(2)-CE detection system 3106 can access one or more databases housed on or accessed by the server device(s) 3102 or elsewhere in the computing system 3000.

FIG. 3B is a block diagram of an exemplary server device 3102 that may be used in connection with the illustrative sequencing system 3000 of FIG. 3A. The server device 3102 may be configured to detect a RHCE*CE-D(2)-CE gene conversion in a nucleic acid sample. The general architecture of the server device 3102 depicted in FIG. 3B includes an arrangement of computer hardware and software components. The server device 3102 may include many more (or fewer) elements than those shown in FIG. 3B. It is not necessary, however, that all of these generally conventional elements be shown in order to provide an enabling disclosure. As illustrated, the server device 3102 includes a processing unit 310, a network interface 320, a computer readable medium drive 330, an input/output device interface 340, a display 350, and an input device 360, all of which may communicate with one another by way of a communication bus. The network interface 320 may provide connectivity to one or more networks or computing systems. The processing unit 310 may thus receive information and instructions from other computing systems or services via a network. The processing unit 310 may also communicate to and from memory 370 and further provide output information for an optional display 350 via the input/output device interface 340. The input/output device interface 340 may also accept input from the optional input device 360, such as a keyboard, mouse, digital pen, microphone, touch screen, gesture recognition system, voice recognition system, gamepad, accelerometer, gyroscope, or other input device.

The memory 370 may contain computer program instructions (grouped as modules or components in some embodiments) that the processing unit 310 executes in order to implement one or more embodiments. The memory 370 generally includes RAM, ROM and/or other persistent, auxiliary or non-transitory computer-readable media. The memory 370 may store an operating system 372 that provides computer program instructions for use by the processing unit 310 in the general administration and operation of the server device 3102. The memory 370 may store a reference genome 373, such as for use by the sequencing application 3110. The memory 370 may further include computer program instructions and other information for implementing aspects of the present disclosure.

For example, in one embodiment, the memory 370 includes a sequencing application 3110, which may include a RHCE*CE-D(2)-CE detection system 3106. The RHCE*CE-D(2)-CE detection system 3106 can perform the methods disclosed herein. In addition, memory 370 may include or communicate with the data store 390 and/or one or more other data stores that store one or more inputs, one or more outputs, and/or one or more results (including intermediate results) of detecting a RHCE*CE-D(2)-CE gene conversion in a nucleic acid sample of the present disclosure, such the sequencing reads, the candidate haplotypes determined, and the variant call (for example, the detection of a RHCE*CE-D(2)-CE gene conversion) determined.

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (such as iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1

The reference genome sequences for RHD and RHCE genes were aligned to each other and all sites with single base differences between the two gene sequences were selected. The positions of those differentiating sites in both the RHD and RHCE genes were stored.

Nucleic acid samples from a diverse population cohort of approximately 3200 individuals were profiled using Illumina® sequencing in a project known as the 1000 Genomes Project. Short sequence reads from the nucleic acid samples were used to determine whether each of the single base differences between the RHD and RHCE were fixed across the population. To do that, a subset of the samples with an estimated combined copy number of four for RHD+RHCE were selected to restrict to those samples without copy number variation. Another set of samples were filtered out if a significant fraction (10% or more) of difference sites between RHD and RHCE had proportions of reads supporting the RHD-specific base (RHD allele) and RHCE-specific base (RHCE allele) inconsistent the assumption that the sample has two copies of each gene (diploid assumption). This step excluded samples where the diploid assumption was broken for either gene, or those samples with large gene conversion events.

Using the subset of filtered samples, each site having a difference between the RHD and RHCE genes was filtered based on how consistently the site had the proportion of reads supporting the RHD allele or the RHCE allele consistent with two copies of each gene across the selected set of samples. Sites were selected as a “fixed differentiating site” if at least 98% of the population samples had similar proportions of reads supporting the RHD allele and the RHCE allele. If these proportions were not met, the site was excluded from the list of fixed differentiating sites. 793 differentiating sites were determined from the RHCE and RHD genes that were single base pair differences in the homologous regions of RHCE and RHD that were found to be fixed in the population (occurring in over 98% of the population).

### Example 2

Sequence reads which aligned to the RHCE or RHD genes from the HG002 reference genome were taken as input. The combined copy number for both RHCE and RHD genes was estimated from the read depth of the reads aligned in the RH genes region, normalized with the read depth of 3000 normalization regions.

A file including two pairs of differentiating sites that flanked the RHCE*CE-D(2)-CE breakpoint sites in the population, and a file including potential haplotypes for the RHCE*CE-D(2)-CE variant were provided as input. Two breakpoints were identified for the RHCE*CE-D(2)-CE gene conversion event, whose corresponding differentiating sites are in positions chr1:25405587 and chr1:25405596 (hg38) for the first breakpoint and chr1:25409676 and chr1:25409958 (hg38) for the second breakpoint.

Candidate haplotypes were formed through a series of extension steps using all reads overlapping the pre-determined differentiating sites between a gene and its paralog, and the total number of haplotypes obtained from the combined copy number of the RHCE and RHD genes. A set of candidate haplotypes were formed from all possible bases at the first pre-determined differentiating site. The haplotypes were then extended at the next differentiating site by considering all reads that could be uniquely assigned to a single candidate haplotype. If these reads supported only a single base at the next differentiating site for a given candidate haplotype, then the haplotype was extended with that base. When a candidate haplotype could be extended by both bases at the next differentiating site, then both possible extended haplotypes were included in the set of candidate haplotypes, growing the set by 1. Subsequent extension steps were performed at neighboring pre-determined differentiating sites until all pre-determined differentiating sites were processed.

To detect a RHCE*CE-D(2)-CE recombinant variant, a haplotype supporting the recombinant variant on the first breakpoint and a haplotype supporting the recombinant variant in proximity of the second breakpoint were identified. Because both the identified candidate haplotypes supported the recombinant variant at the breakpoints, the RHCE*CE-D(2)-CE recombinant variant was detected.

After the RHCE*CE-D(2)-CE gene conversion was detected, the pre-determined differentiating sites that were included in the gene conversion region were evaluated for their copy numbers based on the number of reads containing a RHCE-specific base at the pre-determined differentiating site. If the pre-determined differentiating site bad an estimated RHCE copy number of 0, then a homozygous variant was called for that pre-determined differentiating site. If the pre-determined differentiating site had an estimated RHCE copy number of 1, then a heterozygous variant was called for that pre-determined differentiating site. A VCF-formatted file was saved that included the variant calls

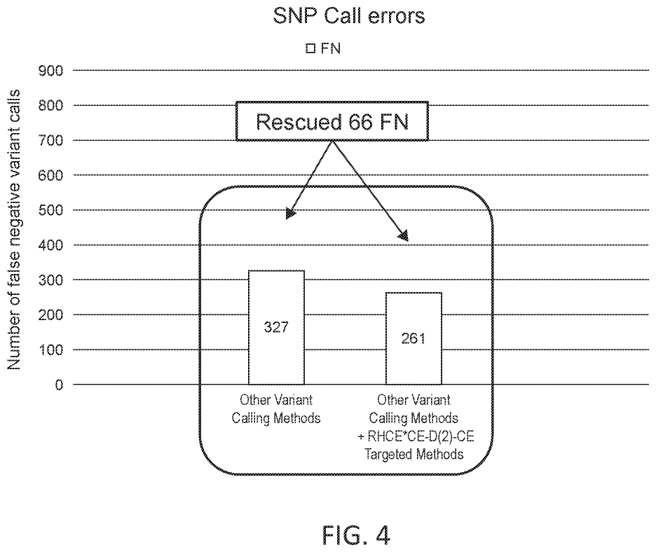

The VCF-formatted file including the variant calls produced by the methods described in this Example, as well as variant calls from other general variant-calling methods, was compared to a “truth VCF” file, which included the variant calls assumed to be the most representative of the HG002 sample. The “truth VCF” file was also compared to a variant call file which was produced using variant calling methods not specific to the RHCE*CE-D(2)-CE gene conversion. As shown in FIG. 4, implementation of the embodiment of the systems and methods for detecting a RHCE*CE-D(2)-CE gene conversion reduced 66 false negative variant calls, meaning that 66 additional SNPs were accurately called as a variant.

### Other Considerations

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, such as a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (such as X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (such as a measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A computer-implemented method of detecting a RHCE*CE-D(2)-CE gene conversion event in a nucleic acid sample, the method comprising:
receiving sequence reads which align to a RHD gene or a RHCE gene;
estimating a combined copy number of a RHD gene and a RHCE gene in the nucleic acid sample;
estimating copy numbers of a RHD-specific base and a RHCE-specific base at each of a plurality of pre-determined differentiating sites of the RHD gene and the RHCE gene; and
calculating a probability of a RHCE*CE-D(2)-CE gene conversion in the nucleic acid sample based on the estimated combined copy number of the RHD gene and RHCE gene and the estimated copy numbers of the RHD-specific and RHCE-specific bases at each of the plurality of pre-determined differentiating sites.

2. The method of claim 1, wherein the RHCE*CE-D(2)-CE gene conversion results in a first breakpoint, and wherein the plurality of pre-determined differentiating sites comprises at least two pre-determined differentiating sites flanking the first breakpoint.

3. The method of claim 2, wherein the method further comprises identifying one or more sequence reads which span the first breakpoint and which include a RHD-specific base at a first pre-determined differentiating site flanking the first breakpoint and a RHCE-specific base at a second pre-determined differentiating site flanking the first breakpoint.

4. The method of claim 3, wherein the RHCE*CE-D(2)-CE gene conversion results in a second breakpoint, wherein the plurality of pre-determined differentiating sites comprises at least two pre-determined differentiating sites flanking the second breakpoint, and wherein the method further comprises identifying one or more sequence reads which span the second breakpoint and which include a RHD-specific base at a first pre-determined differentiating site flanking the second breakpoint and a RHCE-specific base at a second pre-determined differentiating site flanking the second breakpoint.

5. The method of claim 1, wherein estimating copy numbers of a RHD-specific base and a RHCE-specific base at each of a plurality of pre-determined differentiating sites of the RHD and the RHCE genes comprises counting sequence reads which include a RHD-specific base at a pre-determined differentiating site among the plurality of pre-determined differentiating sites, and counting sequence reads which include a RHCE-specific base at the pre-determined differentiating site.

6. The method of claim 5, wherein calculating a probability of a RHCE*CE-D(2)-CE gene conversion comprises estimating a gene-specific copy number at each pre-determined differentiating site of the plurality of pre-determined differentiating sites based on a proportion of sequence reads comprising a RHD-specific or RHCE-specific base at the pre-determined differentiating site multiplied by the estimated combined copy number of the RHD and RHCE genes.

7. The method of claim 6, wherein calculating a probability of a RHCE*CE-D(2)-CE gene conversion includes detecting changes to the gene-specific copy number in consecutive pre-determined differentiating sites.

8. The method of claim 1, wherein estimating a combined copy number of the RHD and RHCE genes comprises counting sequence reads which align to the RHD or RHCE genes.

9. The method of claim 8, wherein estimating the combined copy number comprises normalizing the count of the sequence reads which align to the RHD or RHCE genes and applying a Gaussian Mixture model.

10. The method of claim 1, wherein the method accounts for an opposite orientation of the RHD and the RHCE genes.

11. The method of claim 1, wherein the plurality of pre-determined differentiating sites are identified by a method comprising:
identifying single-base differences between the sequence of the RHD and RHCE genes in a reference sequence, and
selecting, as differentiating sites, single-base differences which are fixed across a population.

12. The method of claim 11, wherein selecting, as differentiating sites, single-base differences which are fixed across a population comprises:
for a plurality of nucleic acid samples, receiving a plurality of sequence reads which align to the RHD and RHCE genes,
for each of the plurality of nucleic acid samples, estimating a gene-specific copy number for the RHD gene and a copy number for the RHCE gene,
selecting a subset of nucleic acid samples of the plurality of nucleic acid samples, wherein the subset of nucleic acid samples comprises nucleic acid samples which are estimated to be diploid for the RHD gene and diploid for the RHCE gene, and
selecting single-base differences which have copy numbers consistent with diploidy for the RHD gene and the RHCE gene in at least 90% of the nucleic acid samples of the subset of nucleic acid samples.

13. The method of claim 1, wherein the method further comprises constructing one or more candidate haplotypes.

14. The method of claim 13, wherein the one or more candidate haplotypes cover a breakpoint region of the RHCE*CE-D(2)-CE gene conversion.

15. The method of claim 13, wherein constructing one or more candidate haplotypes comprises phasing the pre-determined differentiating sites using sequence reads aligned to the RHD or RHCE gene.

16. The method of claim 15, wherein phasing the pre-determined differentiating sites comprises:
constructing one or more candidate haplotypes based on all sequenced bases at a first pre-determined differentiating site, and
extending the one or more candidate haplotypes to a second pre-determined differentiating site by aligning sequence reads of the RHD or RHCE gene.

17. The method of claim 16, wherein the first and second pre-determined differentiating sites flank a breakpoint of the RHCE*CE-D(2)-CE gene conversion.

18. The method of claim 1, further comprising making a variant call at a pre-determined differentiating site of the plurality of pre-determined differentiating sites.

19. The method of claim 1, further comprising making a variant call for the RHCE*CE-D(2)-CE gene conversion.

20. The method of claim 18, wherein the variant call comprises a homozygous or heterozygous variant call.

21. The method of claim 1, further comprising creating a file including a variant call.

22. The method of claim 1, wherein the pre-determined differentiating sites comprise a site corresponding to a position selected from chr1:25405587, chr1:25405596, chr1:25409676, or chr1:25409958 of reference genome hg38.

23. An electronic system for detecting a RHCE*CE-D(2)-CE gene conversion event in a nucleic acid sample, comprising a processor configured to perform a method comprising:
receiving sequence reads which align to a RHD gene or a RHCE gene;
estimating a combined copy number of a RHD gene and a RHCE gene in the nucleic acid sample;
estimating copy numbers of a RHD-specific base and a RHCE-specific base at each of a plurality of pre-determined differentiating sites of the RHD gene and the RHCE gene; and
calculating a probability of a RHCE*CE-D(2)-CE gene conversion in the nucleic acid sample based on the estimated combined copy number of the RHD gene and RHCE gene and the estimated copy numbers of the RHD-specific and RHCE-specific bases at each of the plurality of pre-determined differentiating sites.
